(19) United States
(12) Patent Application Publication
(10) Pub. No.: US 2006/0149392 A1
(43) Pub. Date: Jul. 6, 2006

(54) Biomaterials for guided tissue regeneration and drug delivery

(76) Inventors: Kuo-Huang Hsieh, Taipei (TW); Ken-Yu Chang, Taipei (TW); Chun-Pin Lin, Taipei (TW); Liou Liang Horng, Taipei County (TW); Yung-Hsin Lin, Taipei (TW)

Correspondence Address: BLACKWELL SANDERS PEPER MARTIN LLP, 720 OLIVE STREET, SUITE 2400, ST. LOUIS, MO, 63101, US

(21) Appl. No.: 11/295,303

(22) Filed: Dec. 6, 2005

Related U.S. Application Data
(60) Provisional application No. 60/633,818, filed on Dec. 7, 2004.

(51) Int. Cl. A61F 2/28; C12N 5/08

(52) U.S. Cl. 623/23.58; 623/23.76; 435/396; 424/426

(57) ABSTRACT

The present invention is directed to compositions and methods of using compositions comprising a scaffold with growth factors chemically immobilized thereto for inducing chondrogenesis and/or osteogenesis when implanted in vivo or osteogenesis or chondrogenesis in cultures in vitro. The compositions and methods enhance bone and cartilage growth. Also described are compositions and methods for targeted drug delivery.

### CROSS-REFERENCE TO RELATED APPLICATION

This patent application claims priority to U.S. Provisional Patent Application Ser. No. 60/633,818, filed Dec. 7, 2004.

### BACKGROUND OF THE INVENTION

The present invention relates to compositions and methods of using compositions comprising a scaffold with chemically immobilized growth factors capable of inducing bone, cartilage or other tissue growth when implanted in vivo or in vitro. Also, this invention relates to compositions and methods utilized in target drug delivery and gene therapy.

Physical injury or pathological changes such as removal of a tumor can result in large bone defects in an organism's body. If the bone defect cannot heal in a short period, it is difficult for the bone to recover its original function. Currently, autograft and allograft are the two most common clinical methods of using bone transplantation to cure bone defects. However, these transplantation methods can only be applied to small bone defects due to the restricted and insufficient bone supply. Bone transplantation has much to do with the size and the position of the defect in the human body. In dealing with the cranial defect originating from injury, bone disease, or traumatized jaw bone defect, etc., it is very difficult for an orthopedist or a plastic surgeon to make the surgical operation successful.

Almost all human beings suffer from cavities for a variety of reasons such as over-consumption of sweet foodstuffs and poor dental care. Many also suffer from hypersensitive teeth particularly the middle aged population. It is important for the dental industry to restore, reduce, and prevent these problems. However, despite new technologies, the human population is still suffering from cavities and sensitive teeth.

It is the principal object of the present invention to immobilize single and/or multiple growth factor(s) or other biosignal molecules to a scaffold in order to induce and enhance bone and cartilage growth or other tissues. This invention may also be practiced with other tissues other than bone or cartilage.

It is another object of this invention to utilize the excellent plasticity of a scaffold with chemically immobilized growth factors to overcome the size and position limitations of bone transplantation and increase bone regeneration rate.

It is a further object of this invention to perform in vitro immobilization of osteoblast cells and/or stem cells transfected or transformed with growth factor genes onto a scaffold to increase bone tissue regeneration.

It is another object of this invention to restore tooth bone decay with a scaffold with chemically immobilized growth factors.

It is further object of this invention to perform in vitro co-immobilization of drugs and anti-receptor antibodies to a scaffold target drug delivery to tumor cells.

### SUMMARY OF THE INVENTION

The present invention provides a chondrogenic and/or osteogenic inducing and conductive scaffold with chemically immobilized growth factors. Either soft or hard bone tissue can grow along the surface and/or structure of the scaffold. The scaffold serves as a barrier to restrict the massive migration of undesirable connective tissue cells onto the defect and as a substrate for the migration of desirable osteogenic or chrondogenic cells. The form of the scaffold may be a membrane, sponge-like material, sheet, mesh, plate, screws, plugs, rods, porous forms or any other desirable configuration.

The present invention also provides methods to produce a chondrogenic and/or osteogenic inducing scaffold with chemically immobilized growth factors.

The preferred embodiments of the present invention provide a chondrogenic and/or osteogenic inducing and conductive chitosan membrane or sponge with transforming growth factor and/or bone morphogenetic protein chemically immobilized thereto.

The scaffold with chemically immobilized growth factors described above has been found to induce the differentiation of cells to the osteogenic and/or chondrogenic phenotypes and increase cell construct for bone and cartilage regeneration. These materials have excellent plasticity to overcome the size and position limitations of bone transplantation and increase bone regeneration rate. They also provide excellent binding to the teeth to restore bone decay.

This invention also provides methods to perform in vitro immobilization of osteoblast cells, stem cells, or other cells with growth factor genes to the scaffold, which are then implanted in vivo.

This invention also provides methods and compositions for targeted drug delivery utilizing a scaffold with chemically immobilized drug compounds.

Accordingly, the invention provides compositions and methods for enhancing osteogenic and/or chondrogenic growth and regeneration. The method comprises exposing cells, wounds and/or defects to the scaffold with chemically immobilized growth factors. The invention also provides a process to immobilize growth factors to a scaffold and in vitro immobilization of osteoblast cells or stem cells transfected with growth factor genes onto a scaffold. Drugs can be immobilized onto the scaffold utilizing the same method for target drug delivery.

### BRIEF DESCRIPTION OF THE DRAWINGS

FIG. 1 illustrates immobilized osteoblast cells differentiated from the mesenchymal stem cells loaded with growth factor gene immobilized on a scaffold in place of a bone injury;

FIG. 2 illustrates the drug delivery system of this invention showing localized high drug concentration around a tumor cell provided by immobilized drugs supported on nanoparticles;

FIG. 3 is a photograph of an in vivo diaphyseal defect in the right radius of a rabbit treated by placing a scaffold membrane chemically immobilized with TGF-β1 to cover the defect site;

FIG. 4 shows by X-ray photographs of the in vivo defect site of the right and left radius of a rabbit immediately after injury; and

FIG. 5 shows by X-ray photographs of the in vivo defect site of the right and left radius of a rabbit shown in FIG. 4 after one and one-half months after injury.

### DETAILED DESCRIPTION OF THE INVENTION

Guided Tissue Regeneration (GTR) refers to the placement of a scaffold on the wound area to provide a mechanical barrier to prevent undesirable connective tissue cells from growing. At the same time, the scaffold allows the particular bone or cartilage cells to repopulate the defect and regenerate the desirable tissue. The present invention enhances the ability of a scaffold to regenerate tissue by chemically immobilizing growth factors or other molecules or cells to the scaffold material.

The cell to cell interactions inside the human body that regulate cell growth and cell differentiation play important roles in embryonic development, tissue morphogenesis and maintenance of adult organisms. These interactions are mainly coordinated by two types of proteins: growth factors and cell adhesion molecules. Growth factors are generally secreted as diffusible proteins and can transfer proliferation and differentiation signals by binding to the specific receptors on the target cell membrane. The adhesion molecules assemble the cells into tissues through their adhesive properties. They can regulate cell growth and cell differentiation by transferring signals either directly or via intracellular molecules that are associated with cytoplasmic domains.

Different growth factors bring different effects on the cells. Transforming growth factor-α (TGF-α) is known to play an important role in epidermal wound healing. Transforming growth factor-β (TGF-β, TGF-β1), including bone morphogenetic proteins (BMP, BMP-2), are involved in regulating bone growth and repair. Epidermal growth factors (EGF) have been shown to stimulate cell proliferation. Platelet-derived growth factor (PDGF) is proven as a potent mitogen for cells of osteoblastic lineage and, as a consequence, increases the number of cells expressing the osteoblastic phenotype. Other growth factors include enamel proteins, insulin like growth factor(IGF), fibroblast growth factor(FGF), osteogenic peptide (TP508)Chrysalin®, MW=22311; AGYKDEGKRGDACEGDSGGPFV) and the like.

One with ordinary skill in the art would readily understand what is meant by the term “growth factor.” Growth factors have been studied and characterized for many years, such that the state of knowledge regarding growth factors is highly developed. The amino acid sequence of growth factors can be readily found on many publicly available sequence databases, such as the National Center for Biotechnology Information's Entrez database.

Other than growth factors, cell adhesion or proliferation peptides or proteins may be chemically immobilized onto a scaffold. These peptides or proteins may include fibronectin, collagen, or other peptides or proteins containing the arginine-glycine-aspartate (RGD) sequence.

Some growth factors have been immobilized onto polymers materials. Ito et al. immobilized Epidermal Growth Factor (EGF) onto photoreactive polyallylamine to regulate the cell functions of CHO-ER cell cultured on polyallylamine in vitro. Ito et al. also integrated Insulin onto 2-hydroxyethyl methacrylate/ethyl methacrylate copolymer membranes to increase the growth of fibroblast cells in vitro. However, unlike the preferred embodiment of the present invention, Ito et al. did not chemically immobilize growth factors onto a biodegradable scaffold. Further, Ito et al. did not use a scaffold with chemically immobilized growth factors to promote bone or other tissue regeneration, induction, and/or conduction in vivo. Ito et al. only immobilized growth factors onto a nonbiodegradable polymer material in order to study cell systems in vitro.

Others have incorporated growth factors onto biodegradable polymers through adsorption or absorption, but not through chemical immobilization. For example, Muzzarelli et al. and Park et al. both incorporated various growth factors onto chitosan. However, neither Muzzarelli et al. nor Park et al. chemically immobilized the growth factors—they were merely adsorbed or absorbed onto the polymer material.

In order for the growth factors to be effective, the physical coating, adsorption or inclusion methods, such as Muzzarelli et al. and Park et al., require a large dosage of growth factors which renders their applications very expensive. Further, the large amounts of physically loaded growth factors tend to be released too early in the system, which results in too much waste and ineffective healing. Because of the high release rate of the growth factors, these methods would not be effective in healing large bone or other tissue defects. In order to prevent the early release of the growth factors and achieve effective bone healing and regeneration, the present invention chemically immobilizes the growth factors onto the scaffold materials. The chemically immobilized growth factors effectively and economically provide chondrogenic and/or osteogenic inducing stimulations over an extended period of time to enhance bone and/or cartilage growth.

As used herein, the term “chemically immobilized” means that a chemical bond is formed between the scaffold material and a growth factor or other molecule. The term chemically immobilized may include covalent and ionic chemical bonds, but does not include the mere absorption or adsorption of the growth factors onto or in the polymer scaffold. Most preferably, the growth factors are chemically immobilized via a covalent bond, which ensures the growth factors are not prematurely released.

The term “scaffold” refers to some physical structure which acts as a substrate for the chemically immobilized growth factors. Further, a scaffold provides a mechanical barrier which prevents undesirable connective tissue or other tissue from growing. At the same time, the scaffold provides the structure upon or within which the desired bone or cartilage cells may grow in order to regenerate the desirable tissue. The scaffold may be in the form of a membrane, sponge, gel, or any other desirable configuration that both provides a physical barrier for unwanted cells and a structure upon or within which desirable cells may grow. The actual composition of the scaffold is not critical, as long as it acts as a substrate for the chemically immobilized growth factors, while providing the structure upon or within which new tissue may grow and a barrier to keep undesirable tissue from the defect area.

The scaffold preferably comprises a biodegradable polymer so that no, or very little, of the scaffold remains after the tissue has healed. However, the scaffold may also comprise an inorganic or organic composite. The preferred biodegradable polymer scaffold material used in this invention is chitosan [Poly(1,4-β-D-glucopyranosamine) or (β1,4-poly-glucosamine)]. The term “chitosan” hereinafter refers to chitosan and its derivatives. Chitosan is a linear polysaccharide consisting β(1-4) linked D-glucosamine residues with a variable number of randomly located N-acetyl-glucosamine groups. It shares structure features (N-acetyl-glucosamine moieties) with various glycosaminoglycans and hyalurinic acid that are present in articular cartilage and interact with growth factors. It is commonly prepared from the deacetylation of chitin and the molecular structure is shown in the following figure.

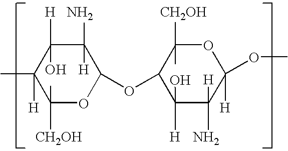

Generally, chitosan and its derivatives have excellent biocompatibility without major fibrous encapsulation. Chitosan enjoys very good biodegradability. The cost of chitosan is inexpensive due to its abundant source. Chitosan is osteoconductive and osteoinductive. Currently, chitosan has widely been applied in health food, artificial skin, medical swab, dialysis membrane, and surgical suture.

Other than chitosan, the scaffold may comprise the following compounds: Poly(ethylene oxide), Poly (lactic acid), Poly(acrylic acid), Poly(vinyl alcohol), Poly(urethane), Poly(N-isopropyl acrylamide), Poly(vinyl pyrrolidone) (PVP), Poly (methacrylic acid), Poly(p-styrene carboxylic acid), Poly(p-styrenesulfonic acid), Poly(vinylsulfonicacid), Poly(ethyleneimine), Poly(vinylamine), Poly(anhydride), Poly(L-lysine), Poly(L-glutamic acid), Poly(gamma-glutamic acid), Poly(carprolactone), Polylactide, Poly(ethylene), Poly(propylene), Poly(glycolide), Poly(lactide-co-glycolide), Poly(amide), Poly(hydroxylacid), Poly(sulfone), Poly(amine), Poly(saccharide), Poly(HEMA), poly(anhydride) and the like. Also, the following natural polymers could be utilized to form the scaffold: Collagen, gelatin, glycosaminoglycans (GAG), Poly (hyaluronic acid), Poly(sodium alginate), Alginate, Hyaluronan, agarose, polyhydroxybutyrate (PHB) and others.

The state of the art of polymer chemistry is well-developed. One with ordinary skill in the art could readily create a polymer scaffold of any desirable configuration, structure, or density. By varying the polymer concentration, solvent concentration, heating temperature, reaction time, and other parameters, one with ordinary skill in the art can create a polymer scaffold with any desired physical characteristic. For example, the polymer scaffold material may be formed into a sponge-like structure of various densities. The porous nature of a sponge-like scaffold allows cells to populate the interior of the scaffold. The scaffold material may also be formed into a membrane or sheet, which could then be wrapped around or otherwise shaped to a bone defect. The scaffold could also be configured as a gel, mesh, plate, screw, plug, or rod. Any conceivable shape or form of the scaffold is within the scope of the present invention.

The polymer may be cross-linked with a cross-lining agent in order to enhance the mechanical strength of the polymer. Possible cross-linking agents may include genipin, glutaraldehyde, tri-polyphosphate (TPP), hydroxyapitite, and any other cross-linking agent known to those skilled in the act.

The polymer may also be combined with hydroxyapatite (HA) to form a polymer/HA compositite. For example, the polymer may be blended with HA. Alternatively, HA may be coated onto the surface of an already formed polymer scaffold. Other materials may also be combined with a polymer to form a composite. For example, calcium phosphate, TCP, hydroxyapatite, collagen, and/or polymethyl methacrylate may be combined with a polymer to form a composite.

The scaffold may also be primarily calcium phosphate, TCP, hydroxyapatite, collagen, polymethyl methacrylate, and/or a mixture of any thereof. Many other calcium or phosphate compounds will work.

Any prior art bone fillers, cements, or scaffolds can be utilized in this invention by chemically immobilizing growth factors to them.

Before chemically immobilizing growth factors onto the scaffold, a spacer molecule may be utilized to overcome the steric problems between the functional groups on the scaffold surface and the large protein molecules of the growth factors. A spacer molecule could provide greater flexibility for the rigid scaffold and allow the immobilized growth factor to move into a suitable orientation and position in order to function properly. Normally, the spacers comprise molecules with less than 10 atoms in length. Common spacers include: succinic acid, diaminodipropylamine (DADPA), 6-aminocaproic acid (6-AC), 1,3-Diamino-2-propanol, 1,6-Diaminohexane (DAH), ethylenediamine (EDA), and the like.

The following are a few processes which chemically immobilize growth factors (designated “R”) onto a scaffold:

Hydroxyl group containing scaffolds could react with amine reactive growth factors according to the following reactions:

(1) Cyanogen Bromide (CNBr)
Scaffold materials carrying hydroxyl functional groups (—OH) could be activated by CNBr to become a very reactive cyanate ester and then coupled with amine reactive growth factors.

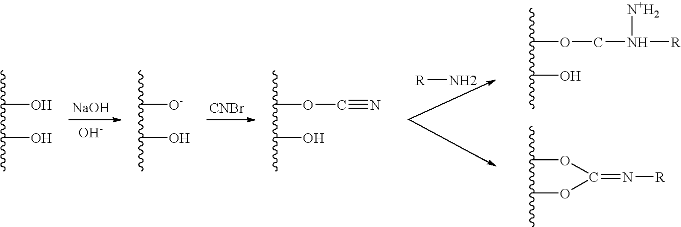

(2) N-Hydroxy Succinimide Esters (NHS Ester)
Scaffold materials carrying hydroxyl function groups could be activated by NHS ester as follows:

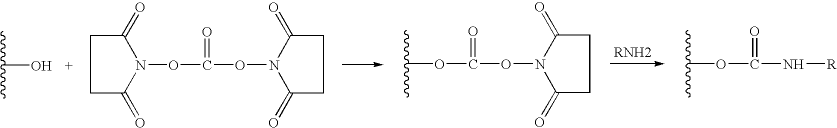

(3) Carboxylic Diimidazole (CDI)
Scaffold materials carrying hydroxyl function groups could be activated by CDI as follows: 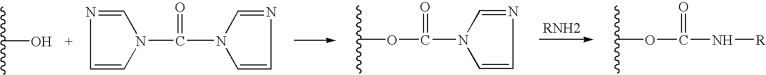
Amine containing scaffold materials could react with amine reactive growth factors as follows:
(1) Glycidol

Amine containing scaffold materials could be transformed to become periodate-activated by reacting with glycidol followed by reacting with amine reactive growth factors.

(2) glutaraldehyde

Primary amine containing scaffold materials could utilize the secondary amine linkage with the terminal formyl groups that are reactive with amine reactive growth factors.

Carboxylic acid containing scaffolds or growth factors could react with amine reactive growth factors or scaffold materials as followings:

(1) Carbodiimide

Carboxylic acid containing scaffold materials or growth factors could be activated by water-soluble-carbodiimide (EDC or WSC) to become activated ester intermediates that can react with amine containing scaffold materials or growth factors.

(1) Carboxylic Diimidazole

Carboxylic acid containing scaffold materials or growth factors could be activated by Carboxylic diimidazole to become activated ester intermediates that can react with amine containing scaffold materials or growth factors.

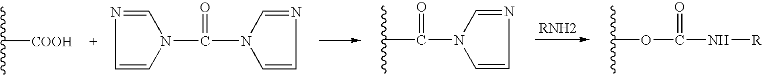

(1) Sulfo-NHS
Carboxylic acid containing scaffold materials or growth factors could be activated by Sulfo-NHS to become activated NHS ester intermediates that can react with amine containing scaffold materials or growth factors.

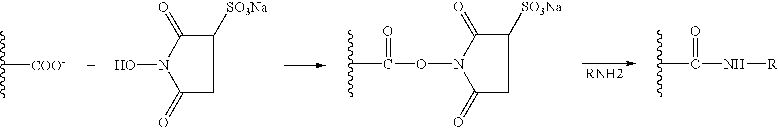

The immunoassays of growth factors may be used to test whether the growth factors are chemically immobilized on the scaffold as well as the bioactivity of the growth factors. Two growth factor immunoassays may be applied in this invention: one is radioimmunoassay (RIA) and the other is enzyme-linked immunosorbent assays (ELISA).

The in vitro osteoblast models can be used to study the bioactivity of the scaffold with chemically immobilized growth factors. There are two kinds of osteoblasts—preosteoblast and osteoblast—used in the models. Growth factors such as TGF, BMP, IGF and bFGF are known to stimulate osteoblast differentiation. In the in vitro culture while the osteoprogenitor cells are placed on the scaffold with chemically immobilized growth factors, the osteoprogenitor cells tend to differentiate into the osteoblast cells and exhibit a higher secretion of alkaline phosphatase (ALP). The cell morphology is also changed. Therefore, one can determine the activity and functionality of the scaffold with chemically immobilized growth factors by measuring type I-collagen or ALP as well as observing the alteration of cell morphology.

The in vivo model is a simple and direct way to study the present invention in bone. This “implant” type study is applied to animal tests to evaluate the effect of a scaffold with chemically immobilized growth factors on bone and cartilage formation. The animal tests provide results that can be linked to humans. In the animal models, the size, skeletal anatomy, similarity to human biology, healing potential, availability, ease of housing, uniformity, and maintenance costs, etc. should all be taken into consideration. By creating a bone defect in an animal followed by practicing the present invention, accelerated bone regeneration can be easily observed.

Bone formation occurs through three coordinated processes: production, maturation, and mineralization of the osteoid matrix. Normally, these processes occur at the same rate so there is equal balance between matrix production and mineralization. Initially, osteoblasts deposit as collagen rapidly without mineralization while producing a thickening osteoid seam. This is followed by an increase in the mineralization rate to catch up with the rate of collagen synthesis. At the final stage, the rate of collagen synthesis decreases and mineralization continues until the osteoid seam is fully mineralized.

For some metabolic bone diseases, the bone is formed very rapidly during the fracture healing and bone development stage. There is no preferential organization of the collagen fibers. This type of bone is called woven bone. Woven bone is characterized by irregular bundles of collagen fibers and many large and extremely numerous osteocytes. Also, there is delayed and disorderly calcification which occurs in irregularly distributed patches. As for the lamellar bone, collagen fibers are preferentially arranged to allow the highest density of collagen per unit volume of tissue. The lamellae can be parallel to each other if deposited along a flat surface (traecular bone and periosteum) or concentric if deposited on a surface surrounding a central blood vessel (cortical bone Haversian system). Woven bone lacks the strength of lamellar bone, and it is for this reason that it is usually replaced by lamellar bone during bone remodeling.

Osteoblast function is regulated by a number of endocrine, paracrine, and autocrine factors. The classical systemic regulators such as vitamin D3, estrogen, RTH, and others are endocrine factors. Paracrine and autocrine factors are regulators secreted by a wide range of interleukins and growth factors. Many growth factors play an important role in bone repair and regeneration. At the initial stage, growth factors such as platelet derived growth factor (PDGF), transforming growth factor beta (TGF-β) and insulin like growth factor (IGF) are involved in the regulation of the cell proliferation. The cells will express the mitogenic growth factors such as TGF-β, PDGF, fibroblast growth factor (FGFs) when they proliferate to a great extent. Among these factors, TGF-β may play a very crucial role in this process because large amounts of it has been found existing in bone matrix. As the cells proliferate for a long period of time, the growth factors for proliferation decrease. Then, cells transform into the differentiation stage while the osteoblast cells synthesize a great amount of bone matrix such as osteocalcin, alkaline phosphate, type I collagen as well as bone morphogenetic proteins (BMPs). BMPs belong to the transforming growth factor-beta superfamily. But it has a different biological effect when compared to TGF-β. For example, BMP-2 is a spontaneous promoter. With the expression of BMP-2, the osteoblast differentiates itself from osteogenic cells. The detailed interaction between growth factors and bone formation and healing is still not known. It is only known that there is a series of processes involved in the regulation of bone formation by growth factors.

Many inherited and acquired diseases, characterized at the genetic level, are known to result from protein deficiencies or defects. Gene-based therapeutic strategies can manage these diseases. Another aspect of this invention involves gene therapy accompanied by the use of a scaffold with chemically immobilized growth factors in order to accelerate bone regeneration.

Viral- and plasmid-based vehicles are often used in gene delivery. The viral delivery system consists of modified, usually nonreplicating, viral genomes carrying a specific transgene. Plasmid-based gene delivery system utilizes a variety of agents (lipids, polymers, peptides) complexed with DNA either encoding a transgene or the naked DNA alone. The most commonly used complexing agents are cationic liposomes and condensing agents such as poly (ethylenimine) (PEI) and poly (L-lysine).

Gene therapy has been used in bone surgery. For example, it has been utilized to achieve posterior intertransverse process fusion in rodents. Riew et al. has applied the gene therapy technique to achieve anterior intradiscal fusion in pigs with the use of minimally invasive techniques. First, the mesenchymal stem cells (MSC) isolated from each of the pigs was transduced with an adenovirus carrying the gene of bone morphogenetic proten-2 (adv-BMP2). Then, a 1 cm³ disc of each the pigs was removed. The discs were then injected with autologous mesenchymal stem cells (MSC) transduced with adv-BMP2. They found the anterior spinal fusion was observed in all disc spaces that had been treated with implantation of Adv-BMP2-transfected stem cells while the discs without any treatment had none. The anterior spinal fusion may result from the BMP-2 protein secreted from the Adv-BMP2-transfected-MSC.

Kadiyala et al. used marrow-derived MSCs to repair a segmental defect in the femur of rats. They found that in the defects treated with MSC-loaded implants, substantial new bone formation occurred at the interface between the host and the implant that lead to a continuous span of bone across the defect. Furthermore, a periosteal callus of bone also present in those samples loaded with MSCs but not with cell-free implants.

It is known that there is a coordinate relationship between bone formation and vasculature. Vasculature provides the orientation for the secretory activity of osteoblasts. As a result, adequate space must be provided for the juxtapositioning of capillaries to the sheets of osteoprogenitor cells in engineering the delivery of osteoprogenitor cells to a reparative site. At an in vivo site, if MSCs aggregate into a dense mass in the absence of intervening capillaries, they differentiate into cartilage. Therefore, it is important to construct a delivery system that has the potential to place sheets of osteoprogenitor cells in a configuration such that the host vasculature can quickly position itself to provide the appropriate cueing for bone development.

The success of such a cell-based method for bone tissue engineering is critically dependent on the development of an appropriate matrix scaffold for cell delivery. The ideal scaffold formulation should possess several properties: (a) the material should foster uniform cell loading, cell division, and retention; (b) the scaffold should support rapid vascular invasion; (c) the scaffold should be designed to orient the formation of new bone in anatomically relevant shapes; (d) the scaffold materials should be resorbed and replaced by new bone as it's formed; (e) the scaffold should encourage osteoconduction with host bone; and (f) it should posses desirable handling properties for the specific clinical indication.

In one embodiment of the present invention, mesenchymal stem cells (MSC) that are transfected with a growth factor-gene are chemically immobilized in vitro onto the surface of a scaffold. The scaffold with chemically immobilized cells can then be applied to the bone defect in order to accelerate bone healing. The mesenchymal stem cells (MSC) that are transfected with a growth factor-gene can secrete the growth factors in greater concentration to induce bone growth and cell differentiation. The sequences of growth factor genes are readily available on NCBI's databases.

One embodiment of the present invention utilizes the immobilization technology described above to chemically immobilize the cells (or MSCs) to the scaffold with hydroxyl, amine, carboxylic acid, and thio or sulfonic functional groups. The coupling reaction of the chemical immobilization of cells (or MSCs) onto a scaffold is made by the specific affinity between the specific receptor on the cell (or MSCs) surface and its antibody (anti-receptor antibody) on the scaffold. First, the specific anti-receptor antibody is immobilized onto the scaffold via the very same reactions between the growth factors and the scaffold having the functional groups mentioned above. For scaffold materials with amine/carboxylic functional groups, they can be coupled with anti-receptor antibody via the very same reactions between the hydroxyl/amine/carboxylic acid/thio or sulfonic functional groups of the antibody with those of the scaffold. In case the scaffold materials are hydroxyl reactive, then, the antibody could be immobilized via the reactions between the hydroxyl groups of the materials and the amine or carboxylic functional groups of the antibody. Afterwards, the in vitro culture of the cells (MSCs) transfected with growth factor genes are immobilized onto the anti-receptor-antibody grafted scaffold through the receptor and anti-receptor-antibody coupling.

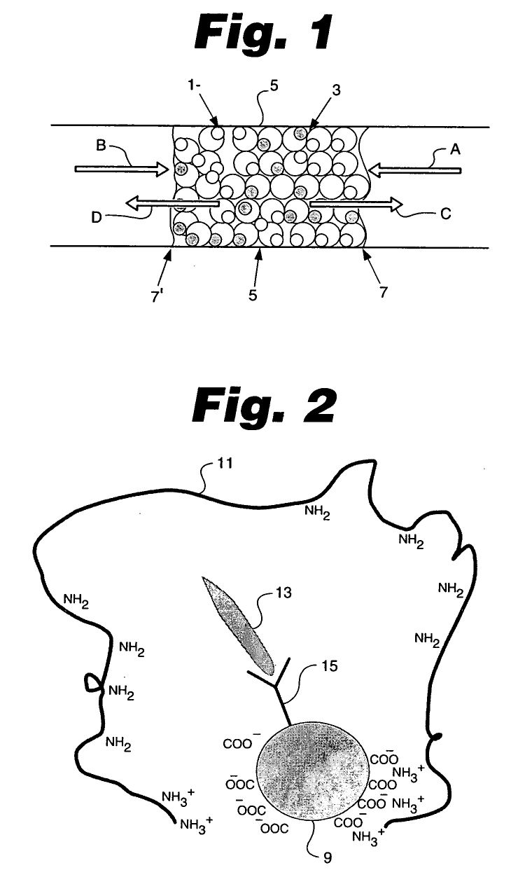

The present invention enables the loading of the growth factor gene transfected or transformed MSCs onto the porous and membrane scaffolds followed by the implantation of the scaffolds onto the bone defect site to accelerate bone healing and regeneration. The chemical immobilization of the growth factors transfected MSCs to a scaffold causing the correct mesengenic process, i.e., correct differentiation path of MSCs in the scaffold. This method directs the multi-directional bone healing and regeneration in the defect site as shown in FIG. 1.

In FIG. 1 there is shown osteoblast cells 1 that are differentiated from MSCs cells 3 transfected or transformed with a growth factor gene. These cells are chemically immobilized onto the scaffold 5. Also shown in FIG. 1 are bone ends 7 and 7′ that define the boundary of the bone injury. Arrows A and B indicate the osteoblast growth of the host bone along the bone end toward the scaffold. Arrows C and D indicate the direction of the growth of MSCs or the growth of the osteoblasts differentiated from the MSCs resulting from the stimulation of growth factors.

In another embodiment of the present invention, drugs are chemically immobilized onto a nanoparticle. The nanoparticle preferably comprises a polymer, most preferably chitosan. There are two mechanisms to chemically immobilize the drugs (usually polypeptides) onto a nanoparticle. They are ionic bonding and covalent bonding. The necessary condition for the ionic bonding between the drugs and nanoparticle is for them to have the dissociate functional groups such as hydroxyl, amine, carboxylic acid, and thio or sulfonic, etc. functional groups. For those nanoparticles without the dissociate functional groups, they can be chemically modified to carry the dissociate functional groups prior to complex with drugs. There are a lot of amine functional groups existing in Chitosan which makes it possible to complex with drugs through ionic bonding. However, due to the dilution or dissociation effect, sometimes the ionic bonding between the drugs and Chitosan is not strong enough to prevent the drugs from early release prior to reaching the target site. Therefore, it is much more desirable to utilize covalent bonding to immobilize the drugs to the nanoparticle for target drug delivery.

A variety of reactions similar to the chemical immobilization of growth factors onto scaffold materials are applied to form chemical immobilization between the drugs and the nanoparticle materials with hydroxyl/amine/carboxylic acid/thio or sulfonic functional groups.

Prior to immobilizing drugs onto a nanoparticle, a spacer molecule may have to be immobilized onto the nanoparticle in order to overcome the steric problems between the functional groups on the nanoparticle surface and the large protein molecules of drugs. For some rigid nanoparticles, a spacer molecule will provide greater flexibility to allow the immobilized ligand to move into position to establish the correct binding orientation. Some of the spacers frequently used are succinic acid, diaminodipropylamine (DADPA), 6-aminocaproic acid (6-AC), 1,3-Diamino-2-propanol, 1,6-Diaminohexane (DAH), ethylenediamine (EDA), etc.

The chemical immobilization of drugs onto polymer materials is achieved through the reaction between the hydroxyl/amine/carboxylic acid/thio or sulfonic functional groups of the drugs and the hydroxyl/amine/carboxylic acid/thio or sulfonic functional groups of the polymer/spacer arm. Some drugs having hydroxyl, amine or carboxylic functional groups could be fixed on the amine functional groups of a polymer directly without a spacer arm.

The target drug delivery to tumor cells is achieved through the co-immobilization of the anti-tumor drugs and the anti-marker-receptor antibody onto a nanoparticle. The methods of the immobilization of the anti-receptor antibody onto a polymer are described previously. This embodiment of the present invention can create localized high drug concentration around the tumor cells targeted. For example, the anti-liver-cancer drugs and anti-marker-receptor antibody of the marker receptor of the liver tumor cells could be chemically immobilized onto amine reactive chitosan nanoparticles as shown in FIG. 2. In FIG. 2 there is shown a tumor cell 9 and a chitosan nanoparticle 11. On particle 11 there is shown anti-marker-receptor antibody 13 attached to a specific marker receptor 15 on tumor cell 9. The drug concentration provided by nanoparticle 11 is shown in FIG. 2 as NH₂⁺ and COO⁻.

Besides target drug delivery, the chemical immobilization of drugs that are useful in bone healing onto materials could have a positive effect on bone or cartilage regeneration to accelerate bone growth and regeneration.

### EXAMPLES

The following examples are not intended to be limiting. They are merely examples to guide one skilled in the art to make and use the present invention.

Preparation of Chitosan Membrane

(I) Preparation of Chitosan Solution

Chitosan powders were agitated in an aqueous acetic acid solution (1 wt %) at 60 C for about 24 hours to provide a final concentration of 4 wt % (however, other concentrations will work).

(II) Preparation of Chitosan Membrane

The chitosan membranes were prepared by the immersion-precipitation method. Glass rods with 3 mm or 4 mm diameter were dipped into the chitosan solution followed by dipping in a 1N NaOH aqueous bath to induce the phase separation. About 20 minutes after dipping, the hollow chitosan membrane formed on the glass rod. Then they were dipped in the de-ionized water for 2 days to neutralize the basic solution. Finally the chitosan membranes were peeled off from the rod.

Chitosan membranes may also be formed into a sheet membrane. A chitosan solution may be spread by a roller to a desired thickness on a plate substrate. Then, it may be immersed in a NaOH solution. The spreading speed, NaOH concentration, thickness of the sheet, material of the plate substrate, immersion time, and other variables may all be varied to achieve the desired results.

(III) Preparation of Chitosan Sponge

The chitosan solutions were put in a cylindrical holder and then frozen by liquid nitrogen or refrigeration in freezer followed by the freeze-drying for 48 hrs.

(IV) Preparation of a Chitosan/HA Composite

Several methods could be applied in the preparation of chitosan/hydroxapatite composite as follows:

Method 1: Blending Method

The chitosan solution (1-4 wt %) in 1 wt % acetic acid and the hydroxyapatite suspended in buffer solution are mixed in various ratios. The solution is then freeze-dried after mixing for a few hours.

Method 2: Coating Apatite onto the Chitosan Scaffold Surface

(ii) Coating of Apatite

First, the chitosan scaffolds (porous or non-porous, 2D or 3D) have to be washed by ethanol. Afterwards, the scaffolds are subjected to a glow discharge treatment in O₂ gas for a few seconds to produce polar groups on chitosan scaffold surface. Then, they are soaked in various concentrations of sodium metasilicate and sodium silicate solutions with various SiO2/Na2O ratios for several hours at 36.5° C. After that, the scaffolds are dried at room temperature and rinsed with distilled water. Finally, they are soaked in different hydroxyapatite concentrations of SBF solutions for apatite crystals to grow on the chitosan scaffolds.

Chemical Immobilization of TGF-beta 1

First, 10 μg TGF-beta 1 was dissolved in 20 c.c. coupling buffer-0.1M sodium phosphate, pH 7.3. Then, 0.4 g EDC and 0.1 g sulfo-NHS were added to the coupling buffer containing dissolved TGF-beta 1 and stirred with a magnetic stirrer for 6 hours at room temperatures. Chitosan hollow membranes were suspended and stirred in the coupling solutions for 72 hrs followed by a series of washing. The chitosan membrane was first washed with the coupling buffer, then water followed by 1M NaCl, and water at the end. After washing, the TGF-beta-1-g-chitoasn membranes were stored in a 0.02% sodium azide preservative.

Although the above example illustrates immobilizing the growth factor onto the scaffold after the scaffold has been formed, the growth factor may also be immobilized before formation of the scaffold structure i.e., before the sponge, membrane, or other structure is formed. The growth factor may be immobilized while the polymer is still in a liquid form. This could allow the growth factor to be evenly distributed throughout the polymer scaffold.

Animal Mode

l Defects, 8-10-mm diaphyseal defect was surgically created in the both radius of rabbits. No fixation was used, as the intact ulna splinted the radius sufficiently. Under general anesthesia and aseptic conditions, a direct anterolateral incision was made over the radius. The periosteum was incised transversally about 2.5 cm from the radiocarpal joint and elevated proximally with a knife. A fine metal band was inserted to make a gap between the radius and the ulna. A transverse osteotomy was performed with an oscillating saw under continuous saline irrigation.

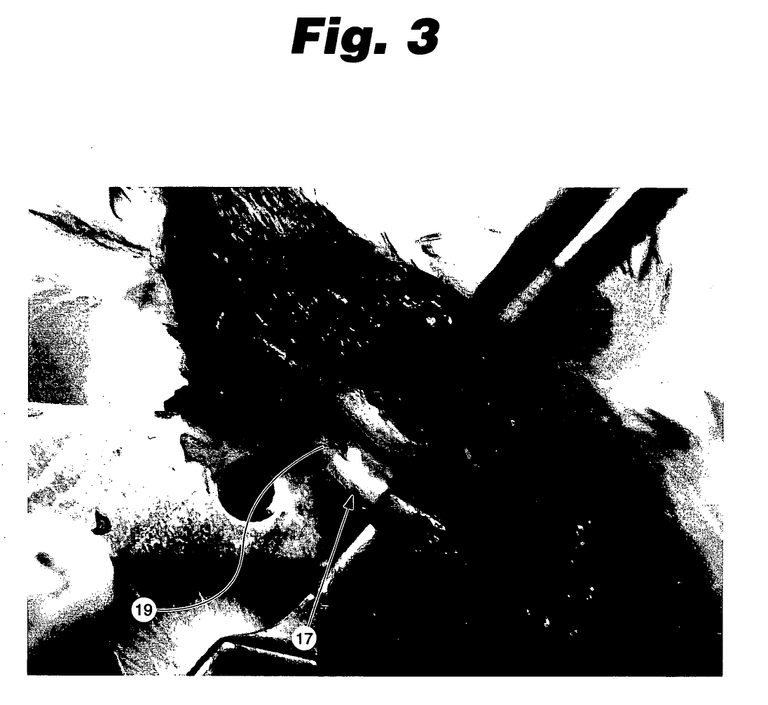

There is shown in FIG. 3 a photographic picture of a chitosan membrane 17 with immobilized TGF-β1 inserted in the right radius bone 19 of a rabbit to cover the defect site of the bone. The injured left radius was not treated and served as the control.

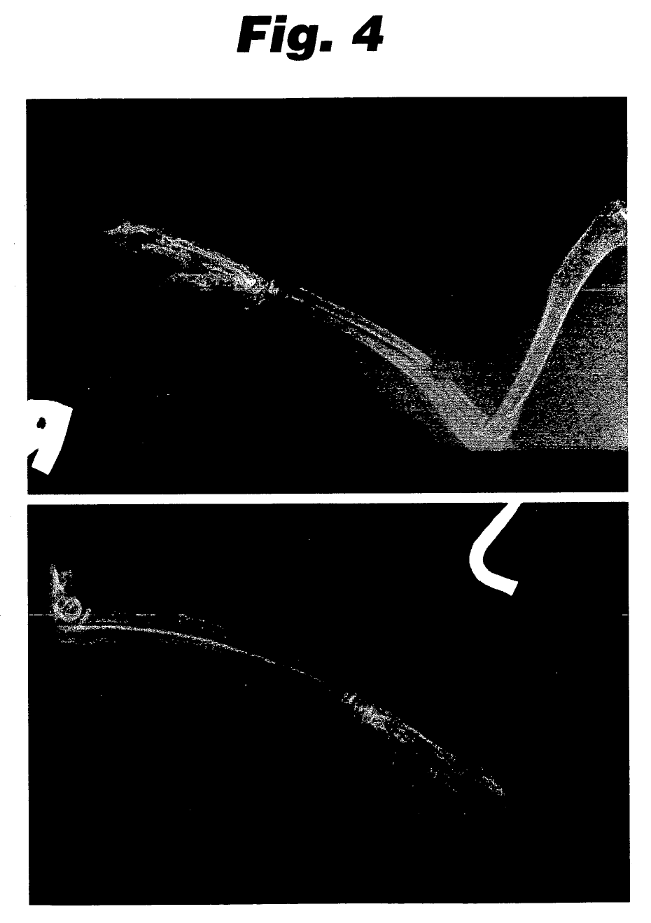

In FIG. 4 there are shown X-Ray photographs showing the injured left and right radii of a rabbit. The X-ray photographs in FIG. 4 are marked R for the right radius that is treated as shown in FIG. 3 while the photograph of FIG. 4 marked L shows the injured radius that was not treated and employed as control for comparison purposes.

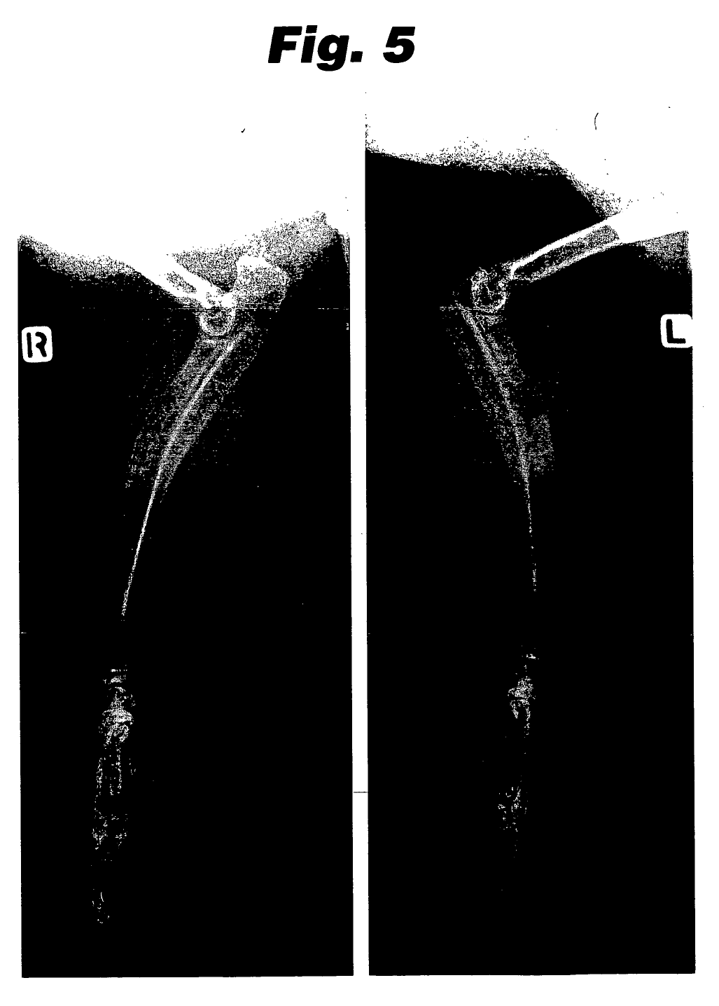

In FIG. 5 there is shown an X-ray photographs of the right (R) radius that was treated as shown in FIG. 3 and the left (L), untreated radius as shown in FIG. 4. The x-ray photographs of FIG. 5 were taken one and one half months after the injury and treatment. The photographs show that the right radius has healed while the injury to the left radius has not healed. These data clearly confirm the fact that the growth factor immobilized chitosan scaffold is a valid chondrogenic/osteogenic inducing and conductive system to accelerate the bone growth and regeneration in bone defect.

We claim:

1. A composition for guided tissue regeneration comprising:
a. A biodegradable scaffold; and
b. At least one growth factor chemically immobilized to said biodegradable scaffold;
wherein said composition is effective for inducing chrondogenesis or osteogenesis in vivo.

2. The composition of claim 1, wherein said biodegradable scaffold comprises a material selected from the group consisting of Poly(ethylene oxide), Poly (lactic acid), Poly(acrylic acid), Poly(vinyl alcohol), Poly(urethane), Poly(N-isopropyl acrylamide), Poly(vinyl pyrrolidone) (PVP), Poly (methacrylic acid), Poly(p-styrene carboxylic acid), Poly(p-styrenesulfonic acid), Poly(vinylsulfonicacid), Poly(ethyleneimine), Poly(vinylamine), Poly(anhydride), Poly(L-lysine), Poly(L-glutamic acid), and Poly(gamma-glutamic acid).

3. The composition of claim 1, wherein said biodegradable scaffold comprises a material selected from the group consisting of Poly(carprolactone), Polylactide, Poly(ethylene), Poly(propylene), Poly(glycolide), Poly(lactide-co-glycolide), Poly(amide), Poly(hydroxylacid), Poly(sulfone), Poly(amine), Poly(saccharide), Poly(HEMA), and poly(anhydride).

4. The composition of claim 1, wherein said biodegradable scaffold comprises a material selected from the group consisting of collagen, gelatin, glycosaminoglycans, poly(hyaluronic acid), poly(sodium alginate), alginate, hyaluronan, agarose and polyhydroxybutyrate.

5. The composition of claim 1, wherein said biodegradable scaffold comprises chitosan.

6. The composition of claim 1, wherein said biodegradable scaffold comprises a composite of chitosan and hydroxyapatite.

7. The composition of claim 1, wherein said biodegradable scaffold comprises chitosan and a cross-linking agent.

8. The composition of claim 1, wherein said biodegradable scaffold comprises chitosan cross-linked with TPP.

9. The composition of claim 1, wherein said at least one growth factor is selected from the group consisting of transforming growth factor-α, transforming growth factor-β1, bone morphogenetic protein, bone morphogenetic protein-2, epidermal growth factor, platelet-derived growth factor, enamel protein, insulin-like growth factor, fibroblast growth factor, and osteogenic peptide.

10. The composition of claim 1 wherein said growth factor is chemically immobilized to said biodegradable scaffold through the use of a spacer molecule selected from the group consisting of succinic acid, diaminodipropylamine (DADPA), 6-aminocaproic acid (6-AC), 1,3-Diamino-2-propanol, 1,6-Diaminohexane (DAH), and ethylenediamine (EDA):

11. A composition for guided tissue regeneration comprising:
a. a scaffold comprising chitosan; and
b. at least one growth factor chemically immobilized to said scaffold;
wherein said composition is effective for inducing chrondogenesis or osteogenesis in vivo.

12. The composition of claim 11 wherein said growth factor is bone morphogenetic protein-2.

13. The composition of claim 11 wherein said growth factor is transforming growth factor-β1.

14. A method of treating a bone defect comprising contacting the area of the bone defect with a scaffold which has at least one growth factor chemically immobilized thereto.

15. The method of claim 14 wherein said scaffold comprises chitosan.

16. The method of claim 14 wherein said growth factor is transforming growth factor-β1.

17. The method of claim 14 wherein said growth factor is bone morphogenetic protein-2.

18. A method treating a bone defect comprising contacting the area of the bone defect with a scaffold comprising chitosan which has transforming growth factor-β1 chemically immobilized thereto.

19. A method treating a bone injury or defect comprising contacting the area of the bone injury with a scaffold comprising chitosan which has bone morphogenetic protein-2 chemically immobilized thereto.

20. A composition for guided tissue regeneration comprising:
a. A biodegradable scaffold; and
b. At least one cell adhesion molecule chemically immobilized to said scaffold;
wherein said composition is effective for inducing chrondogenesis or osteogenesis in vivo.

21. The composition of claim 20 wherein said scaffold comprises chitosan.

22. A composition for guided tissue regeneration comprising:
a. A biodegradable scaffold;
b. a spacer molecule chemically immobilized to said scaffold; and
c. at least one cell adhesion molecule chemically immobilized to said spacer molecule;
wherein said composition is effective for inducing chrondogenesis or osteogenesis in vivo.

23. The composition of claim 22 wherein said scaffold comprises chitosan.

24. A composition for enhancing bone or cartilage growth comprising:
a. a scaffold; and
b. at least one stem cell transfected or transformed with at least one growth factor gene;
wherein said stem cell is chemically immobilized to said scaffold, and further wherein said composition is effective for inducing chrondogenesis or osteogenesis in vivo.

25. The composition of claim 24 wherein said stem cell is chemically immobilized to said scaffold because of the affinity of a cell surface receptor of said stem cell and its antibody, said antibody being chemically immobilized to said scaffold.

26. The composition of claim 24 wherein said scaffold comprises chitosan.

27. A method of treating a bone or cartilage defect comprising contacting said bone defect with a composition comprising:
a. a scaffold; and
b. at least one stem cell transfected or transformed with at least one growth factor gene;
wherein said stem cell is chemically immobilized to said scaffold, and further wherein said composition is effective for inducing chrondogenesis or osteogenesis in vivo.

28. The method of claim 27 wherein said at least one stem cell is chemically immobilized to said scaffold because of the affinity of a cell surface receptor of said stem cell and its antibody, said antibody being chemically immobilized to said scaffold.

29. The method of claim 27 wherein said scaffold comprises chitosan.

30. A method of treating a bone or cartilage defect comprising:
a. transfecting or transforming a growth factor gene into a stem cell;
b. chemically immobilizing an anti-receptor antibody of said stem cell onto a scaffold in vitro;
c. causing said anti-receptor antibody to interact with a cell surface receptor of said stem cell, thereby immobilizing said stem cell onto said scaffold; and
d. implanting in vivo said scaffold having said immobilized stem cell onto said bone or cartilage defect.

31. A composition for targeted drug delivery comprising:
a. a biodegradable nanoparticle;
b. at least one drug chemically immobilized to said biodegradable nanoparticle; and
c. and at least one anti-marker-receptor antibody chemically immobilized to said biodegradable nanoparticle.

32. The composition of claim 31 wherein a spacer molecule is chemically immobilized in between said biodegradable nanoparticle and said drug.

33. A method of treating a tumor comprising administering to an organism a composition comprising:
a. a biodegradable nanoparticle;
b. at least one drug chemically immobilized to said biodegradable nanoparticle; and
c. and at least one anti-tumor-receptor antibody chemically immobilized to said biodegradable nanoparticle.
